(19) United States
(12) Patent Application Publication
(10) Pub. No.: US 2024/0074997 A1
(43) Pub. Date: Mar. 7, 2024

(54) Repurposing Adapalene as a glucocorticoid receptor modulator to treat inflammatory diseases

(71) Applicants: Rene Houtman, Culemborg (NL); Kai Lamottke, Munich (DE)

(72) Inventors: Rene Houtman, Culemborg (NL); Kai Lamottke, Munich (DE)

(21) Appl. No.: 18/348,720

(22) Filed: Jul. 7, 2023

Related U.S. Application Data
(60) Provisional application No. 63/368,098, filed on Jul. 11, 2022.

(51) Int. Cl. A61K 31/192; A61K 9/00

(52) U.S. Cl. CPC A61K 31/192; A61K 9/0014

(57) ABSTRACT

A method of treating an inflammatory disease in a subject in need thereof, comprising administering to the subject an effective amount of a glucocorticoid receptor modulator comprising a therapeutically acceptable salt, solvate, or stereoisomer thereof of Adapalene as well as pharmaceutical compositions comprising Adapalene for the management of these inflammatory diseases.

### BACKGROUND OF THE INVENTION

Glucocorticoids (GC) are steroid hormones mostly generated in the adrenal cortex. The major natural glucocorticoid in humans is cortisol. Key functions of Glucocorticoids include regulation of glucose, protein and lipid metabolism, cell proliferation and differentiation, development, stress response, apoptosis, immune response, and inflammation^{i ii}. Glucocorticoids exert their effects via the glucocorticoid receptor (GR, NR3C1), a well-known transcription factor (TF) from the superfamily of nuclear hormone receptors. In inactive conformation it resides in the cytoplasm bound to the molecular chaperones-heat shock proteins and immunophilins. Upon stimulation by Glucocorticoids, GR undergoes phosphorylation, homodimerization, and translocates to the nucleus. Activation of gene expression (transactivation, TA) requires GR homodimer binding to the palindromic GC responsive elements (GRE) in gene promoters. Negative gene expression regulation (transrepression, TR) is mediated via diverse mechanisms including binding of GR to less conserved negative GREs or by binding of GR monomer to other TFs including pro-inflammatory nuclear factor kappa B (NF-kB), activator protein 1 (AP-1), interferon-regulated factors (IRF), signal transducers and activators of transcription (STATs), thus blocking their activity^{iii iv}. It has been well accepted in the field that TR is an important mechanism underlying therapeutic anti-inflammatory effects of glucocorticoids. At the same time, mediated by GR homodimer TA regulates GR signaling linked to gluconeogenesis, lipid, and protein catabolism, and often mediates the development of atrophic effects in different tissues (such as skin and muscle atrophy) as well as some metabolic adverse effects (hyperglycemia, steroid-induced diabetes)^{v}.

Glucocorticoids are used as therapeutic agents; isolation and crystallization of natural glucocorticoids in 1920-1930s led to the successful synthesis of cortisone in 1947^{vi}. In 1948 the first patient with rheumatoid arthritis was treated with cortisone, and in 1952 hydrocortisone/cortisol was used for the first time to treat atopic dermatitis. Since then, more than 30 glucocorticoids were approved for systemic and topical clinical use, including betamethasone, budesonide, cortisone, dexamethasone (DEX), hydrocortisone, methylprednisolone, prednisolone, prednisone, triamcinolone. Their wide use in clinical practice is based on their strong anti-inflammatory activity and anti-proliferative/pro-apoptotic effects important for anti-cancer activity. Indeed, glucocorticoids inhibit expression of a large set of inflammatory cytokines and chemokines IL-1, IL-2, IL-12, IL-18, TNF-α, INF-γ, GM-CSF, and others, as well as central regulators of cell cycle-cyclins and CDKs,v. Despite a recent successful development of novel anti-inflammatory and anti-cancer therapies targeting specific cytokines and growth factors/pathways, glucocorticoids are still widely used for the treatment of millions of patients with allergies, chronic inflammatory and autoimmune diseases such as asthma, rheumatoid arthritis (RA), ulcerative colitis/Crohn disease, multiple sclerosis, inflammatory and hyperproliferative skin diseases including atopic dermatitis and psoriasis, different skin rashes, itches^{vii viii}. Glucocorticoids are also extensively used for the treatment of different forms of ocular inflammation, macular edema, and macular degeneration and due to their anti-angiogenic properties for prevention of neovascularization in the eye. In addition, they are also an important part of the postoperative patient management and immunosuppressive combination therapies in organ transplant recipients. Synthetic glucocorticoids, such as DEX, are routinely included in chemotherapy protocols of acute lymphoblastic leukemia, chronic lymphocytic leukemia, multiple myeloma, Hodgkin's, and non-Hodgkin's lymphoma^{ix}. In case of epithelial cancers, glucocorticoids are mostly used as a palliative therapy to reduce the adverse effects of chemotherapy: to increase appetite, decrease nausea (for some chemotherapeutic regimens that include cisplatin, glucocorticoids are first line antiemetics), weight loss, reduce fatigue, severe skin rashes typical for epidermal growth factor (EGF) and folate inhibitors. Glucocorticoids are also applied in abatement of pain associated with bone metastasis by inhibiting the secretion of prostaglandins^{x}. Even more, glucocorticoids provided modest therapeutic benefit in early-stage prostate cancer and demonstrated potential for use in ER-positive breast cancers due to GR inhibition of E2-mediated cell proliferation^{xi xii xiii xiv}. Currently glucocorticoids market was estimated for more than $4 billion with the predomination of topical glucocorticoids, which represent up to 60-80% of total dermatological and ophthalmological products sold^{xv}. It is expected that market will continue to grow by ˜3.5-4% yearly due to the increasing incidence of chronic diseases and growing geriatric population preferentially treated with glucocorticoids for inflammatory diseasesv. Unfortunately, chronic treatment with glucocorticoids results in multiple metabolic, atrophic, and other adverse effects that became apparent starting from their early use. The increased understanding of glucocorticoid receptor molecular biology and the mechanisms underlying therapeutic and side effects of glucocorticoids, powered a tremendous effort of pharmaceutical companies and academia directed toward the development of safer glucocorticoid receptor-targeted therapies. There is a need for novel alternative and save glucocorticoid receptor ligands with preserved therapeutic activities but reduced side effects in selected tissues including skin, muscle, and bone through design of cofactor of drug delivery such as topical application.

Topical glucocorticoids are efficacious and widely used drugs in dermatology due to their broad anti-inflammatory activity but are associated with adverse effects preventing long-term clinical use. In particular, development of skin atrophy is an important limiting factor for the use of topical corticosteroids. The mechanism of action of the glucocorticoid receptor has been extensively studied. The activated glucocorticoid receptor can mediate transactivation and transrepression, i.e., activation or repression of gene transcription, respectively. Regulation of target gene transcription may occur via direct binding to glucocorticoid response elements (GREs) on the DNA or by binding of glucocorticoid receptor to other transcription factors. It can also involve interaction with various co-activators and co-repressors. One of the most well-described activities of glucocorticoids is the inhibition of pro-inflammatory gene transcription via inhibition of NF-κB and AP-1 activity^{xvi}. Generally, anti-inflammatory activity of Glucocorticoids was perceived to be associated with transrepression and adverse effects attributed to transactivation. This concept was driving the development of glucocorticoid receptor ligands with dissociated profiles, predominantly favoring transrepression activity. However, this concept turned out to be oversimplified illustrated by limited anti-inflammatory efficacy of dissociated glucocorticoid receptor ligands in mouse models or in clinical trials. Indeed, anti-inflammatory activity is not associated exclusively with transrepression, and neither are adverse effects with transactivation. An example of a full glucocorticoid receptor agonist with a high potency on transrepression and lower potency on transactivation is LEO 134310, a novel non-steroidal glucocorticoid receptor agonist. The profile of LEO 134310 is different from dissociated glucocorticoid receptor agonists designed to possess partial transactivation activity on glucocorticoid receptor. An example of a glucocorticoid receptor modulator which does not include skin atrophy in healthy adults is GW870086 but this compound in contrast to LEO 134310 did not achieve significant clinical efficacy in atopic dermatitis patients^{xvii}.

There are a large number of glucocorticoid receptor modulators targeting the glucocorticoid receptor (NR3C1) approved in one or more territories including: 17-hydroxyprogesterone-caproate, alclometasone-dipropionate, amcinonide, aminoglutethimide, beclomethasone, beclomethasone-dipropionate, betamethasone, betamethasone-acetate, betamethasone-dipropionate, betamethasone-valerate, budesonide, ciclesonide, clobetasol-propionate, clobetasone-butyrate, clocortolone-pivalate, cortisone, cortisone-acetate, deflazacort, desonide, desoximetasone, dexamethasone, dexamethasone-acetate, diflorasone-diacetate, difluprednate, fludroxycortide, flumethasone, flumethasone-pivalate, flunisolide, fluocinolone-acetonide, fluocinonide, fluorometholone, fluorometholone-acetate, fluticasone-propionate, halcinonide, halobetasol-propionate, hydrocortisone, hydrocortisone-acetate, hydrocortisone-butyrate, hydrocortisone-valerate, levonorgestrel, loteprednol, medrysone, megestrol-acetate, meprednisone, methylprednisolone, methylprednisolone-aceponate, methylprednisolone-sodium-succinate, mifepristone, mometasone, mometasone-furoate, piretanide, prednisolone, prednisolone-acetate, prednisolone-sodium-phosphate, prednisone, rimexolone, spironolactone, triamcinolone, and triamcinolone-acetonide.

The modern history of the retinoids begins in 1909, with the discovery of vitamin A in the egg yolk lipid extract. The retinoids group comprises vitamin A (retinol), its natural derivatives (retinaldehyde, retinyl ethers), and many synthetic derivatives^{xviii}. The first retinoids used in the treatment of acne and keratinization diseases were limited by the toxicity and adverse effects of first retinoids generation. Tretinoin was the first retinoid used topically in the treatment of acne, but with a high incidence of adverse effects. Therefore, it has been necessary to optimize these molecules by increasing administration safety. Adapalene discovered at the Centre International de Recherches Dermatologiques first described in 1990^{xix} is a retinoid approved in 1996 by the U.S. Food and Drug Administration (FDA) for the treatment of acne (trade name Differin, producer Galderma) with fewer side effects than tretinoin^{xx}. Adapalene is a stable synthetic derivative of the naphthoic acid which belongs to the class of retinoids^{xx xxi}.

The physiochemical properties of Adapalene are: 6-[3-(1-adamantyl)-4-methoxyphenyl]naphthalene-2-carboxylic acid (IUPAC name), 106685-40-9 (CAS number), D10AD03 (ATC code, retinoids for topical use group), C28H28O3 (molecular formula), 412.52 g/mol, white or almost white powder, >10 g/L soluble at 25° C. in dimethyl sulfoxide, 5 g/L soluble at 25° C. in dimethylformamide and tetrahydrofuran, <1 g/L solubility in ethanol at 25° C. and poor solubility in water, melting point 319-322° C., boiling point 606.3° C. at 760 mmHg, density 1.2 g/cm³, refractive index 1.66, pKa 4.23; 3.99 (strongest acidic), −4.8 (strongest basic), lipophilic parameters log P: 8.04, 8.6; 6.06, 6.47, AlogP: 6.68, XlogP: 7.7, storage temperature 2-8° C.^{xxii}.

Adapalene has the advantage of light stability, inclusive in the presence of benzoyl peroxide in the useful combinations for acne treatment. Adapalene is more stable exposed to light and the oxidation processes than tretinoin^{xxiii}. In a stability study, it was shown that Adapalene is stable in 2 M NaOH solution (boiled for 2 h) and is less stable in acidic condition. Thus, in 0.3 M HCl solution after 10 min of boiling, Adapalene was 28% degraded. In oxidative conditions (heating at 80° C. for 10 min with 30% hydrogen peroxide solution) Adapalene was 30% degraded. The exposure to UV light (254 and 366 nm) degraded 25% of Adapalene in 12 h^{xxiv}

In a 2020 review paper by Rusu et al ^{xxii}, all relevant and the most recent studies regarding new biological effects of Adapalene and new forms with further optimized Adapalene properties were described. This paper shows that Adapalene selectively binds to members of the Retinoic Acid Receptor (RAR) family. Adapalene has a high affinity for RARβ, which are mainly in dermal fibroblasts^{xx} and for RARγ, highly expressed in the epidermis, but is displays lower agonist selectivity towards RARγ than trifarotene^{xxv}. Although the underlying mechanism of action is not fully clarified, topically applied Adapalene modulates keratinization, inflammation, and differentiation of follicular epithelial cells.

Topical pharmaceutical formulations containing Adapalene are well tolerated in the treatment of acne vulgaris. Comparative to other topical retinoids such as Tretinoin, Adapalene has better tolerability^{xxvi}. The reported common side effects of Adapalene are classified as mild adverse reactions and comprise photosensitivity, redness, erythema, dryness, skin discomfort, pruritus, desquamation, and stinging/burning^{xxii}. Two similar pharmaceutical formulas have been prepared regarding tolerability and acceptance (0.1% cream and 0.1% lotion)^{xxvii}. The oral retinoid compounds are known as teratogens. Therefore, these compounds are contraindicated in pregnancy or in women wishing to become pregnant. Adapalene is classified in C category risk (Food and Drug Administration-Pregnancy Categories)^{xxviii}. Adapalene is generally available in two formulations: gel (0,1%, 1%, 3%) and cream (0,1%-1%)

Topical emulgels with Adapalene are modern pharmaceutical forms that can replace gels and creams in a friendlier manner and Adapalene can be loaded into an innovative microemulsion formula that proves to facilitate a trans follicular drug delivery into the skin. Microemulsions including lipid nanoparticles and microemulsions containing natural alkyl polyglucosides can be used to release intradermal Adapalene which have advantages such as greater skin hydration and occlusion effect compared to conventional gel, resulting in improved therapeutic efficacy, and reduction of side effects. For delivery in the hair follicle and upper epidermis, Adapalene can be included into a polymeric nanocarrier such as TyroSphere. Besides the formulations with Adapalene as a unique active pharmaceutical ingredient, the most used combination in acne therapy is Adapalene and benzoyl peroxide (Adapalene 0.1% or 0.3% and benzoyl peroxide 2.5%)^{xxix}. Although Adapalene is known to be administered predominantly topically, a new formulation technique proposes encapsulation of Adapalene within lipid and polymer blended polyester nanoparticles for intravenous administration. This delivery system allows activation of retinoid signaling in the central nervous system (CNS), as demonstrated in an experimental animal model (healthy mice)^{xxx}.

Adapalene is a third-generation retinoid with proven effectiveness in the treatment of acne vulgaris. The mechanism of action is however still not fully known. The mechanism of action and structure activity relationship (SAR) studies suggest the biological potential has not been fully exploited. Therapeutically, Adapalene is an interesting molecule that needs to be additionally evaluated for its biological effects by new studies. Adapalene is also described as a third-generation retinoid for systemic use against bladder cancer and Adapalene has been shows to inhibit prostate cancer cell growth prostate cancer. In addition, Adapalene has been shown in a wound-healing assays where it significantly suppressed the migration ability of RM-1 cells and delayed wound healing time and ratio of acreage in a dose-dependent manner and Adapalene was shown at high doses to decrease the invasiveness of RM-1 cells^{xxxi xxxii}. Adapalene was also shown as a medicament for potential systemic treatment in triple negative breast cancer (TNBC). Studies of Mehraj et al showed that accumulation of reactive oxygen species upon co-treatment with GDC-0941 and Adapalene promoted apoptosis and enhanced sensitivity to GDC in TNBC cells. The findings indicate that Adapalene is a promising therapeutic agent in treating advanced BC tumors and enhance sensitivity to GDC-0741 in inhibiting tumor growth in TNBC models while reducing therapeutic resistance^{xxxiii}.

The patent literature describes among others the use of Adapalene and other antiproliferative drugs that do not have an antagonistic effect with the drug in the preparation of drugs for the prevention or treatment of hematological tumors, especially Adapalene for the prevention or treatment of lymphoma, leukemia, and multiple bone marrow for new uses against tumors. It is a new medicinal use of Adapalene based on the S-phase blocking drug effect for the prevention or treatment of blood system tumors^{xxxiv}.

As described in the present invention, the inventors have surprisingly found that Adapalene acts as a selective glucocorticoid receptor modulator which can be used to treat subjects with an inflammatory disease in need of said selective glucocorticoid receptor modulator.

Despite the continued efforts of pharma as well as academia, the search for selective glucocorticoid receptor modulators, compounds with strong anti-inflammatory or anti-cancer properties but a reduced number or level of side effects, has had limited success so far. Unfortunately, the therapeutic efficacy of such exogenous glucocorticoids is, particularly for systemic use, overshadowed by an unacceptably high number of undesired side effects such as hyperglycemia, osteoporosis, mood swings, and weight gain^{xxxv}.

In recent years the development of successful glucocorticoid receptor modulators has proven challenging with many glucocorticoid receptor modulators showed promising initial results such as LGD-5552, AL-438, MK-5932, GW870086, BI650348, Mapracorat, Dagracorat, AZD5423, AZD7594 and AZD9567 but never got past the pre-clinical stage or failed later in clinical trials^{xxxvi}. Current tools for screening potential selective glucocorticoid receptor modulators suffer from shortcomings and do not always capture the complexity of glucocorticoid receptor signaling. The glucocorticoid receptor is allosterically regulated through interactions with its corresponding response elements and cofactors^{xxxvii} which should be considered when testing potential glucocorticoid receptor modulators through a preferably cofactor recruitment assay.

Coregulators are regulatory proteins that bind to transcription factors, such as nuclear receptors which includes the glucocorticoid receptor, to assist in their transcriptional activity, e.g. by altering local chromatin density and thus accessibility of target gene DNA for RNA-polymerase. Depending on various factors, e.g., coregulator availability, post-translational modifications, and characteristics of the ligand (for instance glucocorticoid receptor modulators), a specific set of coregulators is recruited to the receptor. Knowledge about these interactions is essential for the understanding and prediction of nuclear receptor-mediated effects in different systems, as these coregulators influence the transcriptional outcome of nuclear receptors and act as translators of extra and intracellular signals into gene expression. One method to identify the effect of ligands or drug molecules such as glucocorticoid receptor modulators on the nuclear receptor-coregulator interactions is using microarrays^{xxxviii}. Such a microarray technology described in the art consists of several of many unique coregulator-derived peptides immobilized onto the microarray. Each amino acid peptide contains a nuclear receptor binding motif, including the LXXLL or LXXXIXXXL sequence, in which L represents a Leucine, I represents Isoleucine and X any other amino acid derived from known coactivator or corepressor proteins, respectively.

There is thus an urgent need for novel alternative and save glucocorticoid receptor ligands.

### SUMMARY OF THE INVENTION

As described in the present invention, the inventors have surprisingly found that Adapalene selectively modulates the glucocorticoid receptor activity, which can be used to treat subjects with an inflammatory disease in need of said selective glucocorticoid receptor modulator, Adapalene.

In one embodiment, the methods or uses as taught herein describe a method of treating an inflammatory disease in a subject in need thereof, comprising systemic administering to said subject of an effective amount of a glucocorticoid receptor modulator comprising a therapeutically acceptable salt, solvate, stereoisomer or structural isomers thereof of Adapalene.

In certain embodiments, the methods or uses as taught herein describe a method of treating an inflammatory disease in a subject in need thereof, comprising topical administering to said subject of an effective amount of a glucocorticoid receptor modulator comprising a therapeutically acceptable salt, derivatives, or solvate, stereoisomer or structural isomers thereof.

In yet another embodiment, the methods or uses as taught herein describe the systemic administration for treating an inflammatory disease in a subject in need thereof, comprising the use of an inhaler to administer an effective amount of a glucocorticoid receptor modulator comprising a therapeutically acceptable salt, derivatives, solvate, stereoisomer or structural isomers thereof of Adapalene.

In certain embodiments said inflammatory disease topically treated with said treated effective amount of a glucocorticoid receptor modulator Adapalene, salt or derivatives, stereoisomer or structural isomers thereof inflammatory disease comprises a wound healing disorder.

In certain embodiments of the present invention the glucocorticoid receptor modulator relates to Adapalene and its derivatives, which are chemically related compounds obtained through the structural modification of a functional group in Adapalene. The derivatives can be conveniently prepared in a single reaction step, providing an efficient method for their synthesis according to methods described in the art such as by Kubik S et al^{xxxix}

### BRIEF DESCRIPTION OF THE FIGURES

FIG. 1: Molecular structure of Adapalene.

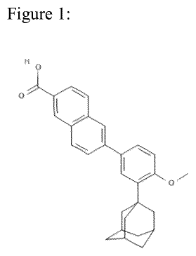

FIG. 2: The glucocorticoid receptor—Ligand Binding Domain (LBD) was stimulated with a concentration series of Adapalene or cortisol, and incubated with coregulator-derived peptide arrays. Binding data as used to construct dose-response curves for the coregulator peptides of which IKBB_277_299, LCOR_41_62, BRD8_255_276, EP300_70_91, NCOA1_1421_1441, NCOA2_679_700, NCOA3_726_747, NRIP1_369_390, PELP1_21_42, NROB2_107_128 and PRGC1_134_154 are depicted as typical examples.

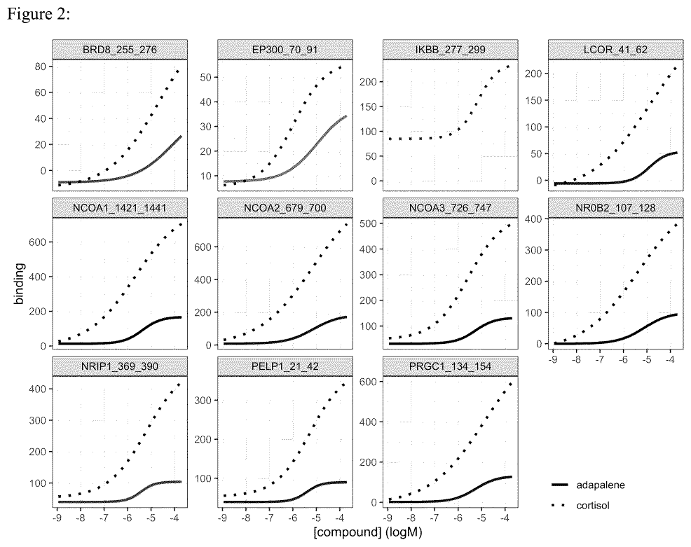

FIG. 3: Individual potencies of cortisol- or Adapalene-induced modulation of glucocorticoid receptor-LBD binding to IKBB_277_299, LCOR_41_62, BRD8_255_276, EP300_70_91, NCOA1_1421_1441, NCOA2_679_700, NCOA3_726_747, NRIP1_369_390, PELP1_21_42, NROB2_107_128 and PRGC1_134_154 were derived as log(EC50) values from the constructed dose-response curved and depicted as boxplot.

FIG. 4: The efficacy (maximal inducible modulation) of Adapalene-induced modulation of glucocorticoid receptor-LBD binding to IKBB_277_299, LCOR_41_62, BRD8_255_276, EP300_70_91, NCOA1_1421_1441, NCOA2_679_700, NCOA3_726_747, NRIP1_369_390, PELP1_21_42, NROB2_107_128 and PRGC1_134_154 relative to that by cortisol was calculated from yₘₐₓ−yₘᵢₙ from constructed dose-response curves and depicted as boxplot.

FIG. 5: Glucocorticoid receptor-LBD binding profile to 11 coregulator peptides upon incubation with by 10 μM Adapalene (grey), 10 μM cortisol (black) or 10 μM cortisol combined with 10 μM Adapalene (dashed line).

FIG. 6: Glucocorticoid receptor-LBD binding profile to 11 coregulator peptides upon incubation with 10 μM Adapalene (black), all-trans retinoic acid (ATRA, dashed line) or solvent only (DMSO, grey).

FIG. 7: Recombinant LBD (upper panel) vs. peripheral blood mononuclear cells (PBMC)-derived full-length (lower panel) protein formulations of Glucocorticoid receptor-binding profile to 11 coregulator peptides upon incubation induced by 10 μM Adapalene (black), cortisol (grey) or solvent only (DMSO, dashed line).

FIG. 8: The number of attracted neutrophils is recorded of clipped zebrafish larvae after treated for 4 hours with solvent only (Veh), 25 μM beclomethasone (Bec) and 1 μM Adapalene (Ada).

### DETAILED DESCRIPTION

All scientific and technical terms used in this application have meanings commonly used in the art unless otherwise specified. As used in this application, the following words or phrases have the meanings specified.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements, or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of ±10% or less, preferably ±5% or less, more preferably ±1% or less, and still more preferably ±0.1% or less of and from the specified value, insofar such variations are appropriate to perform in the disclosed invention. It is to be understood that the value to which the modifier “about” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any >3, >4, >5, >6 or >7 etc. of said members, and up to all said members. In another example, “one or more” or “at least one” may refer to 1, 2, 3, 4, 5, 6, 7 or more.

The term “subject” includes living organisms such as humans, monkeys, cows, sheep, horses, pigs, cattle, goats, dogs, cats, mice, rats, cultured cells, and transgenic species thereof. In a preferred embodiment, the subject is a human.

The term “administering” includes routes of administration which allow the active ingredient of the invention to perform their intended function.

The term “treat” or “treatment” refers to a method of reducing the effects of a disease or condition. Treatment can also refer to a method of reducing the underlying cause of the disease or condition itself rather than just the symptoms. The treatment can be any reduction from native levels and can be, but is not limited to, the complete ablation of the disease, condition, or the symptoms of the disease or condition.

The term “prevent,” “prevention” or “preventing” means inhibition or averting of symptoms associated with the target disease.

The phrase “therapeutically effective amount” refers to that amount of a compound, material, or composition comprising a compound of the present invention, which is effective for producing a desired therapeutic effect, at a reasonable benefit/risk ratio applicable to any medical treatment.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents, and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment but may be referring to the same embodiment. Furthermore, the features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention, and form different embodiments, as would be understood by those in the art. For example, in the appended claims, any of the claimed embodiments can be used in any combination.

The subject of the invention, the new therapeutic use of Adapalene as a glucocorticoid receptor, Adapalene is useful for treating or preventing any inflammatory disease and/or condition, wherein modulation of the glucocorticoid receptor is effective ameliorating symptoms of said diseases. The invention also provides methods for the treatment or prevention of inflammatory disease mediated by Adapalene, a glucocorticoid receptor modulation; preferably inflammatory diseases described as Atopic Dermatitis, Dermatitis Herpetiformis, Dermatomyositis, Juvenile Rheumatoid Arthritis, Psoriasis, Psoriatic Arthritis, Rheumatoid Arthritis.

In certain embodiments, the glucocorticoid receptor modulator, Adapalene, can be used for treating or preventing a wide range of inflammatory diseases, including but not limited to: Acute Lymphocytic Leukemia, Addison's Disease, Adrenal Insufficiency, Adrenocortical Insufficiency, Adrenogenital Syndrome, Allergic Reactions, Allergic Rhinitis, Alopecia, Ankylosing Spondylitis, Aphthous Ulcer, Aspiration Pneumonia, Asthma, Autoimmune Hemolytic Anemia, Berylliosis, Bronchopulmonary Dysplasia, Bullous Pemphigoid, Bursitis, Carpal Tunnel Syndrome, Cerebral Edema, Chorioditis, Chorioretinitis, Cluster Headaches, Cogan's Syndrome, Conjunctivitis, COPD, Corneal Ulcer, Crohn's Disease, Cushing's Syndrome, Dermal Necrosis, Dermatitis, Dermatologic Lesion, Dermatological Disorders, Diffuse Large B-Cell Lymphoma, Duchenne Muscular Dystrophy, Eczema, Epicondylitis, Tennis Elbow, Erythroblastopenia, Evan's Syndrome, Fibromyalgia, Frozen Shoulder, Giant Cell Arteritis, Gout, Gouty Arthritis, Hemolytic Anemia, Herpes Zoster, Herpes Zoster Iridocyclitis, Hypercalcemia of Malignancy, Idiopathic Thrombocytopenic Purpura, IgA Nephropathy, Immunosuppression, Inflammatory Bowel Disease, Interstitial Lung Disease, Iritis, Keloids, Keratitis, Leukemia, Lichen Planus, Lichen Sclerosus, Lichen Simplex Chronicus, Loeffler's Syndrome, Lupus Nephritis, Lymphoma, Meningitis (Haemophilus influenzae, Meningococcal, Pneumococcal), Mixed Connective Tissue Disease, Multiple Myeloma, Multiple Sclerosis, Mycosis Fungoides, Nephrotic Syndrome, Neuralgia, Neuritis, Neurosarcoidosis, Osteoarthritis, Pemphigoid, Pemphigus, Pharyngitis, Plaque Psoriasis, Polymyalgia Rheumatica, Polymyositis/Dermatomyositis, Pulmonary Tuberculosis (including Extrapulmonary and Tuberculous Meningitis), Ramsay Hunt Syndrome, Sarcoidosis, Scleroderma, Seborrheic Dermatitis, Sinusitis, Skin Rash, Synovitis, Systemic Lupus Erythematosus, Systemic Sclerosis, Thrombocytopenia, Thrombocytopenia Idiopathic, Toxic Epidermal Necrolysis, Transverse Myelitis, Ulcerative Colitis (including Active and Ulcerative Proctitis), and Uveitis.

In certain embodiments, the glucocorticoid receptor modulator, Adapalene can be used for treating or preventing rare diseases such as Besnier Boeck (Schaumann) Sarcoïdose and inflammatory rare diseases that are linked or connected with the glucocorticoid receptor including Autoimmune Polyendocrine Syndrome Type II, Complex Regional Pain Syndrome, Dermatomyositis, Granulomatosis with Polyangiitis, Eosinophilic Granulomatosis with Polyangiitis (Churg-Strauss Syndrome) and McCune Albright Syndrome.

For topical applications said glucocorticoid receptor modulator, Adapalene can be formulated in a suitable ointment containing the active component suspended or dissolved in one or more carriers. Carriers for topical administration of the glucocorticoid receptor modulator, Adapalene include, but are not limited to, mineral oil, liquid petrolatum, white petrolatum, propylene glycol, polyoxyethylene, polyoxypropylene compound, emulsifying wax, sugars such as lactose and water. Alternatively, the glucocorticoid receptor modulator, Adapalene can be formulated in a suitable lotion or cream containing the glucocorticoid receptor modulator, Adapalene composition thereof suspended or dissolved in one or more acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Depending on the condition said inflammatory disease may be treated with additional therapeutic agents administered together with the glucocorticoid receptor modulator, Adapalene or a pharmaceutical composition thereof. Those additional agents can be administered sequentially in any order, as part of a multiple dosage regimen, from the glucocorticoid receptor modulator, Adapalene thereof (consecutive or intermittent administration). Alternatively, those agents can be part of a single dosage form, mixed with the glucocorticoid receptor modulator (simultaneous or concurrent administration).

For oral administration, a pharmaceutical composition useful in the present invention can take the form of solutions, suspensions, tablets, pills, capsules, powders, granules, semisolids, sustained release formulations, elixirs, aerosols, and the like. Tablets containing various excipients such as sodium citrate, calcium carbonate and calcium phosphate are employed along with various disintegrants such as starch, preferably potato or tapioca starch, and certain complex silicates, together with binding agents such as polyvinylpyrrolidone, sucrose, gelatin, and acacia. Additionally, lubricating agents such as magnesium stearate, sodium lauryl sulfate and talc are often very useful for tableting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the glucocorticoid receptor modulator of this invention can be combined with various sweetening agents, flavoring agents, coloring agents, emulsifying agents and/or suspending agents, as well as such diluents as water, ethanol, propylene glycol, glycerin and various like combinations thereof.

The term “parenteral” as used herein refers to modes of administration which include intravenous, intramuscular, intraperitoneal, intrasternal, subcutaneous, intramedullary, and intraarticular injection and infusion. A glucocorticoid receptor modulator, Adapalene for parenteral injection can comprise pharmaceutically acceptable sterile aqueous or nonaqueous solutions, dispersions, suspensions, or emulsions as well as sterile powders for reconstitution into sterile injectable solutions or dispersions just prior to use. Aqueous solutions are especially suitable for intravenous, intramuscular, subcutaneous, and intraperitoneal injection purposes. In this connection, the sterile aqueous media employed are all readily obtainable by standard techniques well-known to those skilled in the art. Examples of suitable aqueous and nonaqueous carriers, diluents, solvents, or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity can be maintained, for example, using coating materials such as lecithin, by the maintenance of the required particle size in the case of dispersions, and using surfactants.

The glucocorticoid receptor modulator, Adapalene useful in the present invention can also contain adjuvants such as, but not limited to, preservatives, wetting agents, emulsifying agents, and dispersing agents. Prevention of the action of microorganisms can be ensured by the inclusion of various antibacterial and antifungal agents, such as for example, paraben, chlorobutanol, phenol sorbic acid, and the like. It can also be desirable to include isotonic agents such as sugars, sodium chloride, and the like. Prolonged absorption of the injectable glucocorticoid receptor modulator, Adapalene can be brought about by the inclusion of agents that delay absorption such as aluminum monostearate and gelatin.

Administration by slow infusion is particularly useful when intrathecal or epidural routes are employed. Several implantable or body-mountable pumps useful in delivering compound at a regulated rate are known in the art. Suspensions, in addition to the glucocorticoid receptor modulator, Adapalene can contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof. For purposes of transdermal (e.g., topical) administration, dilute sterile, aqueous, or partially aqueous solutions (usually in about 0.1% to 5% concentration), otherwise similar to the above parenteral solutions, are prepared.

The glucocorticoid receptor modulator, Adapalene useful in the present invention can also be administered by nasal aerosol or inhalation. Such compositions are prepared according to techniques well-known in the art of glucocorticoid receptor modulator, Adapalene formulations and can be prepared as solutions in saline, employing benzyl alcohol or other suitable preservatives, absorption promoters to enhance bioavailability, fluorocarbons, and/or other conventional solubilizing or dispersing agents.

Compositions for rectal or vaginal administration are preferably suppositories which can be prepared by mixing the glucocorticoid receptor modulator, Adapalene with suitable non-irritating excipients or carriers such as cocoa butter, polyethylene glycol or a suppository wax which are solid at room temperature but liquid at body temperature and therefore melt in the rectum or vaginal cavity and release the drugs. Other pharmaceutically acceptable carriers include, but are not limited to, a non-toxic solid, semisolid or liquid filler, diluent, encapsulating material or formulation auxiliary of any type, including but not limited to ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Solid pharmaceutical excipients include, but are not limited to, starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients can be selected from glycerol, propylene glycol, water, ethanol, and various oils, including those of petroleum, animal, vegetable, or synthetic origin, e.g., peanut oil, soybean oil, mineral oil, sesame oil, etc. Preferred liquid carriers, particularly for injectable solutions, include water, saline, aqueous dextrose, and glycols. Methods of preparing various pharmaceutical compositions with a certain amount of glucocorticoid receptor modulator are known, or will be apparent considering this disclosure, to those skilled in this art.

In some embodiment, the effective amount of a thin film of a glucocorticoid receptor modulator, Adapalene containing cream, foam, gel, lotion, ointment or otherwise topical epicutaneous application described herein with an effective glucocorticoid receptor modular, Adapalene concentration that ranges from 0.1% to 1.0% preferable 0.3%. to the skin or body surface area affected by said inflammatory disease.

In some embodiments, the effective amount of a glucocorticoid receptor modulator, Adapalene is used systemically.

### EXAMPLES

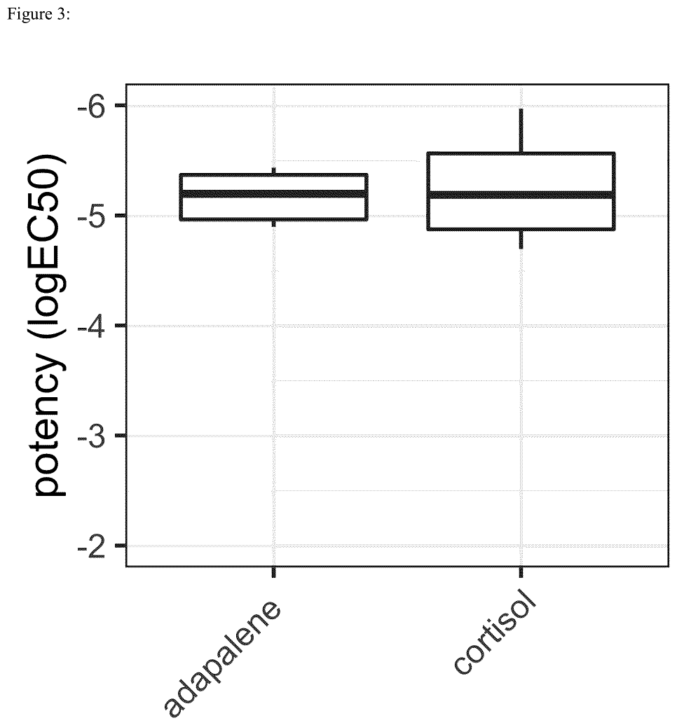

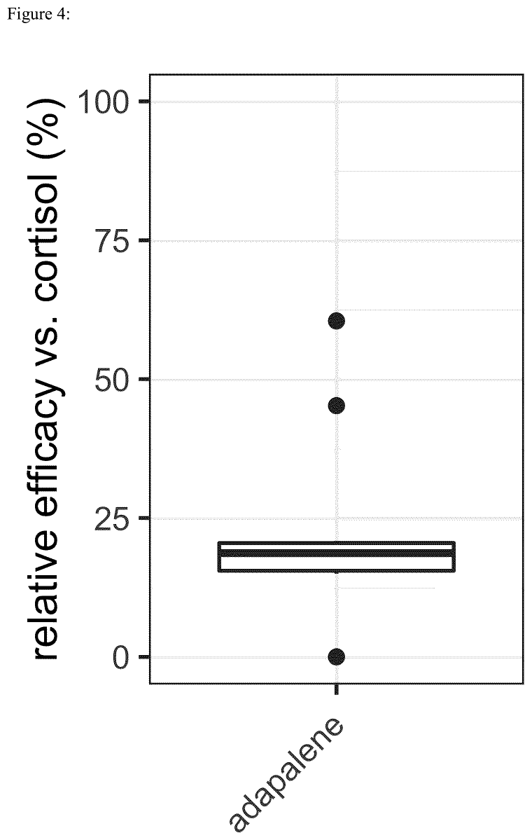

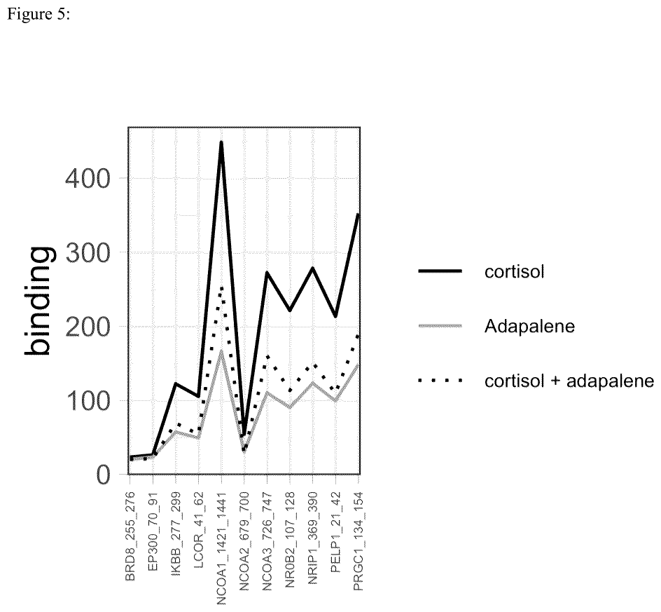

EXAMPLE 1: Interactions between the glucocorticoid receptor ligand binding domain (LBD) and coregulator-derived peptides were determined using the method as described by Zalachoras et al 2009^{xl}. Each peptide array consisting of 11 coregulator peptides as described in Table 1 were incubated with a reaction mixture of 1 nM purified glucocorticoid receptor recombinant human protein, ligand binding domain, (Thermo Fisher Scientific, cat #A15668), ALEXA488-conjugated anti-Glutathione S-Transferase (GST) antibody and buffer F (PV4689, A-11131, and PV4547; Invitrogen). In short, glucocorticoid receptor LBD was incubated with a 12-point concentration series (3-fold dilution in 2% DMSO) of either Adapalene or cortisol starting at a concentration of 0.05 mM compared to solvent. Incubation was performed at 20° C. on the peptide array, using one two arrays per condition. glucocorticoid receptor binding to each peptide on the array, reflected by fluorescent signal, was quantified by image analysis using R-based software (Precision Medicine Lab). The 11 coregulator peptides used represent a range of coregulator nuclear receptor-boxes known to interact with the glucocorticoid receptor and other nuclear receptors, which sequence details, UniProt Knowledge Base accession numbers are shown in Table 1. The dose-response curves were generated for glucocorticoid receptor-LBD treated with Adapalene and cortisol are depicted in FIG. 3 which shows equal potencies of 10 μM for Adapalene and 10 μM for cortisol. On average, the efficacy of Adapalene (the maximal inducible effect of a compound) in modulation of glucocorticoid receptor-coregulator binding as (depicted in FIG. 4) was found to be 20% of (5-fold lower than) that of cortisol. Adapalene is a partial glucocorticoid receptor agonist compared to cortisol with equal potency. Glucocorticoid receptor binding to each peptide on the array, reflected by fluorescent signal, was quantified by image analysis using R-based software (Precision Medicine Lab). The glucocorticoid receptor binding profiles in the presence of cortisol, Adapalene or cortisol and Adapalene, are shown in FIG. 5.

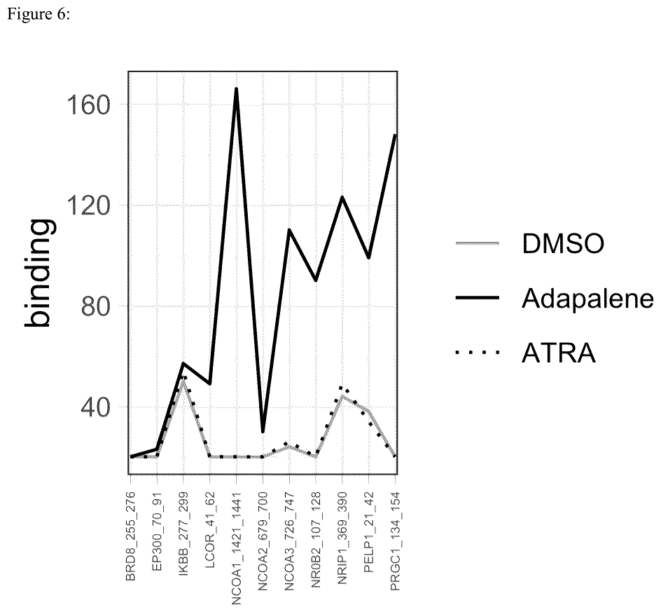

EXAMPLE 2: Adapalene and all-trans retinoic acid (ATRA) are both retinoids and Retinoid X Receptor (RXR) modulators. In this example, the interaction profile between the glucocorticoid receptor ligand binding domain and 11 coregulator-derived peptides and modulation thereof by Adapalene or ATRA are tested as described in Experiment 1. An array with coregulator peptides were incubated with a reaction mixture of 1 nM purified glucocorticoid receptor recombinant human protein, ligand binding domain (Thermo Fisher Scientific, cat #A15668), ALEXA488-conjugated anti-GST antibody and buffer F (PV4689, A-11131, and PV4547; Invitrogen). For ligand induced peptide interaction profiling experiments a concentration of 0.1 mM of Adapalene or ATRA compared to solvent (2% DMSO in water) were used. Incubation was performed at 20° C. on the peptide array. Glucocorticoid receptor binding to each peptide on the array, reflected by fluorescent signal, was quantified by image analysis using R-based software (Precision Medicine Lab). The glucocorticoid receptor binding profiles in the presence of Adapalene or ATRA, or solvent only, are shown in FIG. 6. The results show that Adapalene acts as a glucocorticoid receptor agonist whereas ATRA does not display any glucocorticoid receptor-modulating effect.

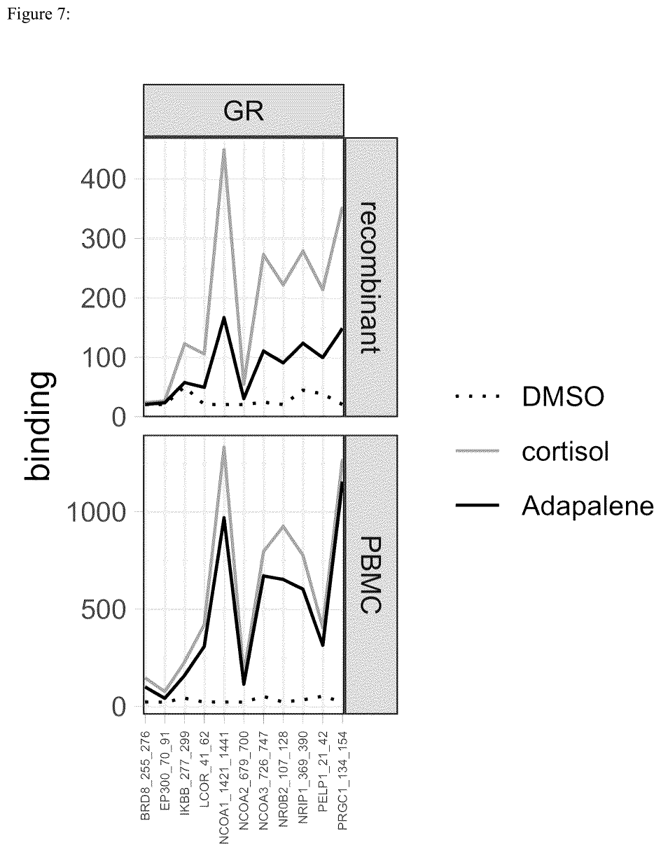

EXAMPLE 3: In this experiment, the interaction profile between the PBMC-derived full-length glucocorticoid receptor or recombinant ligand binding domain and 11 coregulator-derived peptides and modulation thereof by Adapalene or cortisol are compared. The use of glucocorticoid receptor LBD was as described in Experiment 1. In short, we used a reaction mixture of 1 nM purified glucocorticoid receptor recombinant human protein, ligand binding domain (Thermo Fisher Scientific, cat #A15668), ALEXA488-conjugated anti-GST antibody and buffer F (PV4689, A-11131, and PV4547; Invitrogen). As a source for full length glucocorticoid receptor, we first isolated PBMC from buffy coat by Ficoll gradient according to manufacturer protocol. Next, cell extracts from isolated PBMC were prepared as described previously^{xli}. For screening of coregulator binding we used a reaction mixture of PBMC extract (input empirically optimized), 10 nM glucocorticoid receptor-antibody (SC-393232, Santa Cruz), 40 nM ALEXA488-conjugated anti-mouse antibody (A21202 Life Technologies) and buffer F (PV4547; Invitrogen). For ligand induced peptide interaction profiling experiments a concentration of 0.1 mM of Adapalene or cortisol compared to solvent (2% DMSO in water) were used. Incubation was performed at 20° C. on the peptide array. Glucocorticoid receptor binding to each peptide on the array, reflected by fluorescent signal, was quantified by image analysis using R-based software (Precision Medicine Lab). The binding profiles induced by Adapalene or cortisol on either recombinant glucocorticoid receptor LBD or full-length glucocorticoid receptor isolated from human PBMCs are shown in FIG. 7. The results show that Adapalene acts as a partial glucocorticoid receptor agonist on the glucocorticoid receptor LBD and almost full glucocorticoid receptor agonist on full-length glucocorticoid receptor isolated from PBMCs.

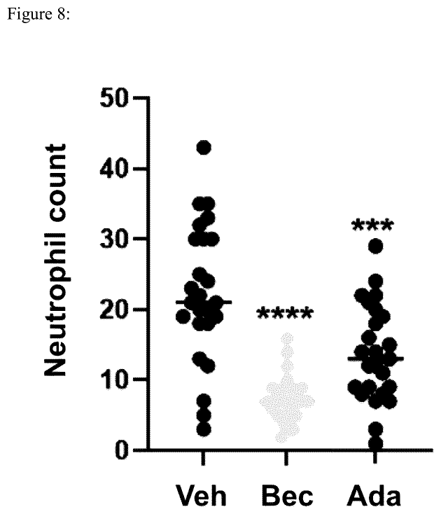

EXAMPLE 4: In this experiment, the inhibitory effect of Adapalene in a Zebrafish inflammation model is tested. The details of this model have been described previously (Xie, Y., Meijer, A. H. & Schaaf, M. J. M. Modeling Inflammation in Zebrafish for the Development of Anti-inflammatory Drugs. Front. Cell Dev. Biol. 8, 620984 (2021)). In short, a zebrafish model with fluorescently labeled neutrophils is used. The tip of the tail fin of 3 day old zebrafish larvae are clipped. As a result, a local inflammatory response is initiated and evokes to leukocyte migration towards the wound. The number of attracted neutrophils is recorded and serves as parameters for inflammation. Clipped zebrafish larvae (FIG. 8) were treated for 4 hours with solvent only (Veh), 25 μM beclomethasone (an anti-inflammatory reference corticosteroid, Beh) and 1 μM Adapalene (Ada) and subsequently neutrophil presence was recorded. Both beclomethasone and Adapalene display significant anti-inflammatory action as illustrated by decrease of the number of attracted neutrophils to the site of the wound. (Significance ***: p<0.001, as determined by Student's t-Test vs. Vehicle)

TABLE 1

| | | Peptide | Uniprot |
|---|---|---|---|
| No | ProbeID | Sequence | Accession |
| 1 | IKBB_277_299 | LGSAMLRPNPILARLLRAHGAP | Q15653 |
| 2 | LCOR_41_62 | TSPTAATTQNPVLSKLLMADQD | Q96JN0 |
| 3 | BRD8_255_276 | VAASPAASGAPTLSRLLEAGPT | Q9H0E9 |
| 4 | EP300_70_91 | MVQDAASKHKQLSELLRSGSSP | Q09472 |
| 5 | NCOA1_1421_ | TSGPQTPQAQQKSLLQQLLTE | Q15788 |
| | 1441 | | |
| 6 | NCOA2_679_700 | HGTSLKEKHKILHRLLQDSSSP | Q15596 |
| 7 | NCOA3_726_747 | QLSPKKKENNALLRYLLDRDDP | Q9Y6Q9 |
| 8 | NRIP1_369_390 | NNIKQAANNSLLLHLLKSQTIP | P48552 |
| 9 | PELP1_21_42 | TGGLSAVSSGPRLRLLLLESVS | Q8IZL8 |
| 10 | NR0B2_107_128 | FEVAEAPVPSILKKILLEEPSS | Q15466 |
| 11 | PRGC1_134_154 | PPQEAEEPSLLKKLLLAPANT | Q9UBK2 |

### REFERENCES

ⁱ Kadmiel M, Cidlowski J A. Glucocorticoid receptor signaling in health and disease. Trends Pharmacol Sci. 2013 September; 34(9):518-30. doi: 10.1016/j.tips.2013.07.003. Epub 2013 Aug. 14. PMID: 23953592, PMCID: PMC3951203.
ⁱⁱ Ramamoorthy S, Cidlowski J A. Corticosteroids: Mechanisms of Action in Health and Disease. Rheum Dis Clin North Am. 2016 February; 42(1):15-31, vii. doi: 10.1016/j.rdc.2015.08.002. PMID: 26611548; PMCID: PMC4662771.
ⁱⁱⁱ Ramamoorthy S, Cidlowski J A. Ligand-induced repression of the glucocorticoid receptor gene is mediated by an NCoR1 repression complex formed by long-range chromatin interactions with intragenic glucocorticoid response elements. Mol Cell Biol. 2013 May; 33(9):1711-22. doi: 10.1128/MCB.01151-12. Epub 2013 Feb. 19. PMID: 23428870; PMCID. PMC3624172.
^{iv} Pufall M A. Glucocorticoids and Cancer. Adv Exp Med Biol. 2015; 872:315-33. doi: 10.1007/978-1-4939-2895-8_14. PMID: 26216001; PMCID: PMC5546099.
^{v} Lesovaya E A, Chudakova D, Baida G, Zhidkova E M, Kirsanov K I, Yakubovskaya M G, Budunova IV. The long winding road to the safer glucocorticoid receptor (GR) targeting therapies. Oncotarget. 2022 Feb. 18; 13:408-424. doi: 10.18632/oncotarget.28191. PMID: 35198100; PMCID: PMC8858080
^{vi} Granner D K, Wang J C, Yamamoto K R. Regulatory Actions of Glucocorticoid Hormones: From Organisms to Mechanisms. Adv Exp Med Biol. 2015; 872:3-31. doi: 10.1007/978-1-4939-2895-8_1. PMID: 26215988.
^{vii} Reuter K C, Grunwitz C R, Kaminski B M, Steinhilber D, Radeke H H, Stein J. Selective glucocorticoid receptor agonists for the treatment of inflammatory bowel disease: studies in mice with acute trinitrobenzene sulfonic acid colitis. J Pharmacol Exp Ther. 2012 April; 341(1):68-80. doi: 10.1124/jpet.111.183947. Epub 2012 Jan. 10. PMID: 22235147.
^{viii} Coenraads P J. Hand eczema. N Engl J Med. 2012 Nov. 8; 367(19):1829-37. doi: 10.1056/NEJ.Mcp1104084. PMID: 23134383.
^{ix} Holland J F, Freireich E J. Emil Frei III, 1924-2013. J Clin Oncol. 2013 Oct. 20; 31(30):3727-30. doi: 10.1200/JCO.2013.51.8126. Epub 2013 Sep. 9. PMID: 24019548.
^{x} Lin K T, Wang L H. New dimension of glucocorticoids in cancer treatment. Steroids. 2016 July; 111:84-88. doi: 10.1016/j.steroids.2016.02.019. Epub 2016 Feb. 27. PMID: 26930575.
^{xi} Auchus R J, Yu M K, Nguyen S, Mundle S D. Use of prednisone with abiraterone acetate in metastatic castration-resistant prostate cancer. Oncologist. 2014 December; 19(12):1231-40. doi: 10.1634/theoncologist.2014-0167. Epub 2014 Oct. 31. PMID: 25361624; PMCID: PMC4257741.
^{xii} West D C, Pan D. Tonsing-Carter E Y. Hernandez K M. Pierce C F. Styke S C, Bowie K R. Garcia T I, Kocherginsky M, Conzen S D. GR and ER Coactivation Alters the Expression of Differentiation Genes and Associates with Improved ER+ Breast Cancer Outcome. Mol Cancer Res. 2016 August; 14(8):707-19. doi: 10.1158/1541-7786.MCR-15-0433. Epub 2016 May 2. PMID: 27141101; PMCID: PMC5008962.
^{xiii} Tonsing-Carter E, Hernandez K M, Kim C R. Harkless R V. Oh A, Bowie K R, West-Szymanski D C, Betancourt-Ponce M A. Green B D, Lastra R R, Fleming G F, Chandarlapaty S, Conzen S D. Glucocorticoid receptor modulation decreases ER-positive breast cancer cell proliferation and suppresses wild-type and mutant ER chromatin association. Breast Cancer Res. 2019 Jul. 24; 21(1):82. doi: 10.1186/s13058-019-1164-6. PMID: 31340854; PMCID: PMC6651939.
^{xiv} Zhidkova E M, Lylova E S, Savinkova A V, Mertsalov S A, Kirsanov K I, Belitsky G A, Yakubovskaya M G. Lesovaya E A. A Brief Overview of the Paradoxical Role of Glucocorticoids in Breast Cancer. Breast Cancer (Auckl). 2020 Dec. 20; 14:1178223420974667. doi: 10.1177/1178223420974667. PMID: 33424228; PMCID: PMC7755940.
^{xv} Lili L N, Klopot A. Readhead B, Baida G, Dudley J T, Budunova I. Transcriptomic Network Interactions in Human Skin Treated with Topical Glucocorticoid Clobetasol Propionate. J Invest Dermatol. 2019 November; 139(11):2281-2291. doi: 10.1016/j.jid.2019.04.021. Epub 2019 Jun. 25. PMID: 31247200; PMCID: PMC6814545.
^{xvi}De Bosscher K, Haegeman G. Minireview: latest perspectives on antiinflammatory actions of glucocorticoids. Mol Endocrinol. 2009 March; 23(3):281-91. doi: 10.1210/me.2008-0283. Epub 2008 Dec. 18. PMID: 19095768; PMCID: PMC5428155.
^{xvii}Dölle S, Hielscher N, Bareille P J, Hardes K, Robertson J, Worm M. Clinical efficacy and tolerability of a novel selective corticosteroid in atopic dermatitis—two randomised controlled trials. Skin Pharmacol Physiol. 2015; 28(3):159-66. doi: 10.1159/000367696. Epub 2015 Jan. 20. PMID: 25614148.
^{xviii} Mukherjee S, Date A, Patravale V. Korting H C, Roeder A. Weindl G. Retinoids in the treatment of skin aging: an overview of clinical efficacy and safety. Clin Interv Aging. 2006; 1(4):327-48. doi: 10.2147/ciia.2006.1.4.327. PMID: 18046911; PMCID: PMC2699641.
^{xix} Hensby C, Cavey D, Bouclier M, Chatelus A, Algate D, Eustache J, Shroot B. The in vivo and in vitro anti-inflammatory activity of CD271: a new retinoid-like modulator of cell differentiation. Agents Actions. 1990 January; 29(1-2):56-8. doi: 10.1007/BF01964720. PMID: 2109510.
^{xx} Waugh J, Noble S. Scott Li. Adapalene: a review of its use in the treatment of acne vulgaris. Drugs. 2004; 64(13):1465-78. doi: 10.2165/00003495-200464130-00005. PMID: 15212561.
^{xxi} Piskin S, Uzunali E. A review of the use of adapalene for the treatment of acne vulgaris. Ther Clin Risk Manag. 2007 August; 3(4):621-4. PMID: 18472984; PMCID: PMC2374937.
^{xxii} Rusu A, Tanase C, Pascu G A, Todoran N. Recent Advances Regarding the Therapeutic Potential of Adapalene. Pharmaceuticals (Base1). 2020 Aug. 28; 13(9):217. doi: 10.3390/ph13090217. PMID: 32872149; PMCID: PMC7558148.
^{xxiii} Irby C E, Yentzer B A, Feldman S R. A review of adapalene in the treatment of acne vulgaris. J Adolesc Health. 2008 November; 43(5):421-4. doi: 10.1016/j.jadohealth.2008.06.005. PMID: 18848668.
^{xxiv} Burkhart C., Morrell D., Goldsmith L. Dermatological Pharmacology. In: Brunton L. L., Chabner B. A., Knollmann B. C., editors. Goodman & Gilman's: The Pharmacological Basis of Therapeutics. McGraw-Hill Education; New York, NY, USA: 2015
^{xxv} Scott L J. Trifarotene: First Approval. Drugs. 2019 November; 79(17):1905-1909. doi: 10.1007/s40265-019-01218-6. PMID: 31713811.
^{xxvi} Kolli S S. Pecone D, Pona A. Cline A, Feldman S R. Topical Retinoids in Acne Vulgaris: A Systematic Review. Am J Clin Dermatol. 2019 June; 20(3):345-365. doi: 10.1007/s40257-019-00423-z. PMID: 30674002.
^{xxvii} Herndon J H Jr. Stephens T J, Trookman N S, Rizer R L, Preston N. Caveney S. Gottschalk R W. A comparison of the tolerability of adapalene 0.1% cream and adapalene 0.1% lotion in healthy individuals. Skinmed. 2012 May-June; 10(3):13642. PMID: 22779096.
^{xxviii} Differin (Adapalene) Gel Label Revised 02/2012. [(accessed on May 9, 2022)]; Available online https://www.accessdata.fda.gov/drugsatfda_docs/label/2012/021753s004lbl.pdf
^{xxix} Latter G, Grice J E. Mohammed Y, Roberts M S, Benson HAE. Targeted Topical Delivery of Retinoids in the Management of Acne Vulgaris: Current Formulations and Novel Delivery Systems. Pharmaceutics. 2019 Sep. 24; 11(10):490. doi: 10.3390/pharmaceutics11100490. PMID: 31554188; PMCID: PMC6835300.
^{xxx} Medina D X, Chung E P, Teague C D. Bowser R. Sirianni R W. Intravenously Administered, Retinoid Activating Nanoparticles Increase Lifespan and Reduce Neurodegeneration in the SOD1^{G93A} Mouse Model of ALS. Front Bioeng Biotechnol. 2020 Mar. 27; 8:224. doi: 10.3389/fbioe.2020.00224. PMID: 32292776; PMCID: PMC7118553.
^{xxxi} Jeevan Ghosalkar et al. Abstract 2316: In vitro and in vivo anticancer potential of adapalene, a third-generation retinoid against bladder cancer Cancer Res (2018) 78 (13_Supplement): 2316.
^{xxxii} Nong H B, Zhang Y N, Bai Y G, Zhang Q, Liu M, Zhou Q, Shi Z H, Zeng G F, Zong S H. Adapalene Inhibits Prostate Cancer Cell Proliferation In Vitro and In Vivo by Inducing DNA Damage, S-phase Cell Cycle Arrest, and Apoptosis. Front Pharmacol. 2022 Feb. 22; 13:801624. doi: 10.3389/fphar.2022.801624. PMID: 35273495; PMCID: PMC8902295.
^{xxxiii} Mehraj U, Wani N A, Hamid A, Alkhanani M, Almilaibary A, Mir M A. Adapalene inhibits the growth of triple-negative breast cancer cells by S-phase arrest and potentiates the antitumor efficacy of GDC-0941. Front Pharmacol. 2022 Aug. 8; 13:958443. doi: 10.3389/fphar.2022.958443. PMID: 36003501; PMCID: PMC9393306.
^{xxxiv} Patent application CN111329851
^{xxxv} Schäcke H, Döcke WD, Asadullah K. Mechanisms involved in the side effects of glucocorticoids. Pharmacol Ther. 2002 October; 96(1):23-43. doi: 10.1016/s0163-7258(02)00297-8. PMID: 12441176.
^{xxxvi} Van Moortel L, Gevaert K, De Bosscher K. Improved Glucocorticoid Receptor Ligands: Fantastic Beasts, but How to Find Them? Front Endocrinol (Lausanne). 2020 Sep. 24; 11:559673. doi: 10.3389/fendo.2020.559673. PMID: 33071974; PMCID: PMC7541956.
^{xxxvii} Desmet S J, Bougarne N, Van Moortel L, De Cauwer L, Thommis J, Vuylsteke M, Ratman D, Houtman R, Tavernier J, De Bosscher K. Compound A influences gene regulation of the Dexamethasone-activated glucocorticoid receptor by alternative cofactor recruitment. Sci Rep. 2017 Aug. 14; 7(1):8063. doi: 10.1038/s41598-017-07941-y. PMID: 28808239; PMCID: PMC5556032.
^{xxxviii} Desmet S J, Dejager L, Clarisse D, Thommis J, Melchers D, Bastiaensen N, Ruijtenbeek R, Beck I M, Libert C, Houtman R, Meijer O C, De Bosscher K. Cofactor profiling of the glucocorticoid receptor from a cellular environment. Methods Mol Biol. 2014; 1204:83-94. doi: 10.1007/978-1-4939-1346-6_8. PMID: 25182763.
^{xxxix} Kubik, S. (2005). Derivate. In F. Böckler, B. Dill, G. Eisenbrand, F. Faupel, B. Fugmann, T. Gamse, R. Matissek, G. Pohnert, A. Rühling, S. Schmidt, & G. Sprenger (Eds.), RÖMPP. Stuttgart: Thieme Gruppe. Retrieved Jul. 4, 2023, from https://roempp.thieme.de/lexicon/RD-04-00600
^{xl} Zalachoras I, Houtman R, Atucha E, Devos R, Tijssen A M, Hu P, Lockey P M, Datson N A, Belanoff J K, Lucassen P J, Joëls M. de Kloet E R, Roozendaal B, Hunt H. Meijer O C. Differential targeting of brain stress circuits with a selective glucocorticoid receptor modulator. Proc Natl Acad Sci USA. 2013 May 7; 110(19):7910-5. doi: 10.1073/pnas.1219411110. Epub 2013 Apr. 23. PMID: 23613579; PMCID: PMC3651427.
^{xli} Houtman R, de Leeuw R, Rondaij M, Melchers D. Verwoerd D, Ruijtenbeek R. Martens J W, Neefjes J, Michalides R. Serine-305 phosphorylation modulates estrogen receptor alpha binding to a coregulator peptide array, with potential application in predicting responses to tamoxifen. Mol Cancer Ther. 2012 April; 11(4):805-16. doi: 10.1158/1535-7163.MCT-11-0855. Epub 2012 Feb. 7. PMID: 22319200.

1. A method of treating an inflammatory disease in a subject in need thereof, comprising systemic administering to said subject of an effective amount of a glucocorticoid receptor modulator comprising a therapeutically acceptable salt, solvate, stereoisomer or structural isomers thereof of Adapalene.

2. A method of treating an inflammatory disease in a subject in need thereof, comprising topical administering to said subject of an effective amount of a glucocorticoid receptor modulator comprising a therapeutically acceptable salt, solvate, or stereoisomer thereof of Adapalene.

3. The systemic administration to said subject of claim 1 comprising an inhaler.

4. The inflammatory disease of claim 2 comprises a wound healing disorder.
